(19) United States
(12) Patent Application Publication
(10) Pub. No.: US 2022/0087843 A1
(43) Pub. Date: Mar. 24, 2022

(54) DELIVERY SYSTEM FOR DEPLOYING A SELF-EXPANDING TUBE, AND METHOD OF DEPLOYING A SELF-EXPANDING TUBE

(71) Applicant: Oxford Endovascular Ltd., Oxford (GB)

(72) Inventors: Andrew MOOR, Northleach (GB); George HSIEH, Southampton (GB); Duncan KEEBLE, Newbury (GB)

(73) Assignee: Oxford Endovascular Ltd., Oxford (GB)

(21) Appl. No.: 17/517,548

(22) Filed: Nov. 2, 2021

Related U.S. Application Data
(63) Continuation of application No. PCT/GB2020/051332, filed on Jun. 3, 2020.

(30) Foreign Application Priority Data
Jun. 14, 2019 (GB) 1908576.0

(51) Int. Cl. A61F 2/966

(52) U.S. Cl. CPC A61F 2/966; A61F 2002/823

(57) ABSTRACT

In one arrangement, there is provided a delivery system for deploying a self-expanding tube into a blood vessel, comprising a tubular member configured for insertion into the blood vessel, an elongate body extending within a lumen of the tubular member, and a self-expanding tube positioned radially between the tubular member and the elongate body. The delivery system is configured to operate in a deployment mode in which there is relative movement longitudinally between the elongate body and a portion of the self-expanding tube that remains in engagement with the elongate body during retraction of the elongate body in use after at least a portion of the self-expanding tube has been deployed, retraction of the elongate body comprising longitudinal movement towards a proximal end of the delivery system of the elongate body relative to the tubular member.

### CROSS-REFERENCE TO RELATED APPLICATIONS

This application is a continuation of International Patent Application Number PCT/GB2020/051332 filed Jun. 3, 2020, which claims the benefit of priority to GB 1908576.0 filed Jun. 14, 2019, the contents of which are incorporated herein by reference in their entireties.

### TECHNICAL FIELD

The present invention relates to systems and methods for deploying a self-expanding tube, particularly for use in redirecting blood flow away from an aneurysmal sac.

### BACKGROUND

An intracranial aneurysm is a weak region in the wall of an artery in the brain, where dilation or ballooning of the arterial wall may occur. Histologically, decreases in the tunica media, the middle muscular layer of the artery, and the internal elastic lamina cause structural defects. These defects, combined with hemodynamic factors, lead to aneurysmal out-pouchings. Intracranial aneurysms are quite common diseases with a prevalence ranging from one to five percent among adult population according to autopsy studies. In the US alone, ten to twelve million people may have intracranial aneurysms.

Current methods for treating intracranial aneurysms include surgical clipping and endovascular coiling. In the surgical clipping method, the skull of the patient is opened, and a surgical clip is placed across the neck of the aneurysm to stop blood from flowing into the aneurysm sac. The risk of this method is relatively high, especially for elderly or medically complicated patients. Endovascular coiling is a less invasive method involving placement of one or more coils, delivered through a catheter, into the aneurysm until the sac of the aneurysm is completely packed with coils. It helps to trigger a thrombus inside the aneurysm. Although endovascular coiling is deemed to be safer than surgical clipping, it has its own limitations. First, after the aneurysm is filled with the coils, it will remain its original size. As a result, the pressure on the surrounding tissue exerted by the aneurysm will not be removed. Second, this procedure is not very effective for wide-necked aneurysms, where the coil is likely to protrude into the parent vessels. This problem may be mitigated by using a stent in combination with coiling embolization, but the procedure is difficult and time-consuming.

Using a self-expanding tube, sometimes referred to as a stent, alone to treat the aneurysm is a promising way to avoid the problems stated above. In this method, a tube with an area of relatively low porosity is placed across the aneurysm neck in such a way as to redirect blood flow away from the sac and trigger formation of a thrombus within the aneurysm. Because the aneurysm solidifies naturally on itself, there is less danger of its rupture. Furthermore, because no coil is involved in this method, the aneurysm will gradually shrink as the thrombus is absorbed. Consequently, the pressure applied on the surrounding tissue can be removed. It is difficult, however, to deploy a self-expanding tube optimally in this context. The tube has to be flexible enough to pass through and adapt to the shape of the very tortuous blood vessels in the brain while at the same time providing sufficient coverage (low porosity) to redirect blood flow away from the aneurysm to an adequate extent. The tube needs to be deployed reliably and controllably, with a minimal risk of damage to the tube or surrounding tissue.

Some current methods for deploying self-expanding tubes, or stents, into blood vessels involve the use of a catheter and guide wire, with the stent in its compressed form wrapped around the guide wire inside the catheter. Once the catheter is positioned in approximately the correct position relative to the aneurysm, the stent is deployed from the catheter by extending the guide wire beyond the end of the catheter.

The deployed stent expands radially and contracts longitudinally relative to its compressed state, and therefore the guide wire must typically be extended beyond the end of the catheter significantly further than the final position of the end of the stent. This introduces a risk that the guide wire may damage the blood vessel or temporarily obstruct perforator blood vessels during deployment if it extends far beyond the final stent position, particularly if used in the very narrow and tortuous blood vessels of the brain.

### SUMMARY

It is an object of the invention to provide apparatus and methods for improving the process of deploying a self-expanding tube, particularly in the context of treating an intracranial aneurysm. In particular, it is an object of the invention to provide an apparatus and method for deploying a self-expanding tube, wherein the risk of damage to blood vessels by a guide wire of the delivery system is reduced. It is a further object of the invention to provide an apparatus and method which allows for the more accurate deployment of a self-expanding tube into a blood vessel.

According to a first aspect of the invention, there is provided a delivery system for deploying a self-expanding tube into a blood vessel, comprising a tubular member configured for insertion into the blood vessel, an elongate body extending within a lumen of the tubular member, and a self-expanding tube positioned radially between the tubular member and the elongate body, wherein the delivery system is configured to operate in a deployment mode in which a first longitudinal engagement force acting between the self-expanding tube and the tubular member and a second longitudinal engagement force acting between the self-expanding tube and the elongate body are such that there is substantially no relative movement longitudinally between the elongate body and any portion of the self-expanding tube that remains in engagement with the elongate body during deployment of the self-expanding tube in use, deployment of the self-expanding tube comprising longitudinal movement towards a proximal end of the delivery system of the tubular member relative to the elongate body, and there is relative movement longitudinally between the elongate body and a portion of the self-expanding tube that remains in engagement with the elongate body during retraction of the elongate body in use after at least a portion of the self-expanding tube has been deployed, retraction of the elongate body comprising longitudinal movement towards a proximal end of the delivery system of the elongate body relative to the tubular member.

By configuring the delivery system such that the elongate body (guide wire) can move relative to the self-expanding tube when the guide wire is retracted, an incremental deployment method is made possible, wherein the guide wire is prevented from extending a significant distance beyond the end of the delivery system during deployment of the self-expanding tube.

In an embodiment, the first longitudinal engagement force is larger, with respect to opposing retraction of the self-expanding tube relative to the tubular member, after a portion of the self-expanding tube has been deployed out of the tubular member than when none of the self-expanding tube has been deployed out of the tubular member. This configuration allows the self-expanding tube to be more easily positioned within the delivery catheter, by allowing it to be moved easily both distally and proximally prior to deployment.

In an embodiment, the self-expanding tube is configured to self expand from a radially contracted state to a radially expanded state in a process involving longitudinal shortening of the self-expanding tube relative to a longitudinal axis of the tubular member, and the larger first longitudinal engagement force is achieved by engagement of a radially expanded and longitudinally contracted portion of the self-expanding tube with a distal end of the tubular member. By configuring the delivery system such that the movement of the guide wire relative to the self-expanding tube is enabled by deployment of a portion of the self-expanding tube, it is ensured that the self-expanding tube and elongate body can move freely back and forth in the proximal and distal directions inside the tubular member prior to the start of deployment.

In an embodiment, a distal end of the elongate body comprises a distal engagement member configured to detachably engage with the self-expanding tube. Providing an engagement member allows the self-expanding tube to be retrieved even after it has been substantially deployed, in case of any error in deployment or other event during the deployment procedure, which necessitates aborting the insertion of the self-expanding tube entirely.

In an embodiment, over at least 50% of the length of the self-expanding tube, at least a portion of the self-expanding tube engages outwardly with the tubular member and inwardly with the elongate body. Engagement of the self-expanding tube with the tubular member and elongate body over a majority of its length spreads the engagement force applied to the self-expanding tube over a greater length. This reduces the chance of damage to the self-expanding tube occurring from too large a force being applied to a small region of the tube.

In an embodiment, either or both of a composition and surface texture of the inner surface of the tubular member is uniform over a length in which the tubular member is in contact with the self-expanding tube. In an embodiment, either or both of a composition and surface texture of the outer surface of the elongate body is uniform over a length in which the elongate body is in contact with the self-expanding tube. These embodiments ensure consistent behaviour during all stages of deployment and reduce the chance of damage to the self-expanding tube.

In an embodiment, the self-expanding tube has a porosity of less than 85% when deployed. This embodiment allows the self-expanding tube to redirect blood flow away from an aneurysm effectively once deployed.

According to a second aspect of the invention, there is provided a delivery system for deploying a self-expanding tube into a blood vessel, comprising a tubular member configured for insertion into the blood vessel, an elongate body extending within a lumen of the tubular member, a self-expanding tube positioned radially between the tubular member and the elongate body, and a retaining member configured to selectively apply a retaining force longitudinally to a proximal region of the self-expanding tube, wherein the delivery system is configured to operate in a retraction mode in which the application of the retaining force allows relative movement longitudinally between the elongate body and a portion of the self-expanding tube that remains in engagement with the elongate body during longitudinal movement of the self-expanding tube in a proximal direction relative to the elongate body.

In some situations, it may be necessary to recover the self-expanding tube after it has been at least partially deployed, for example if the self-expanding tube has been deployed in an incorrect location. Another example is if the self-expanding tube moves substantially in the blood vessel after deployment was started, such that deployment must be begun again to ensure the self-expanding tube is correctly placed. In such a situation, it is advantageous to provide a delivery system with an operating mode where an additional force can be applied to the self-expanding tube to draw it back into the tubular member by sliding it relative to the elongate body. This means the self-expanding tube can be retracted and redeployed even where the elongate body does not extend fully under the deployed portion of the expanding tube due to the use of a deployment mechanism such as that described above.

In an embodiment, in the retraction mode, the application of the retaining force is such that there is substantially no relative movement longitudinally between the elongate body and any portion of the self-expanding tube that remains in engagement with the elongate body during longitudinal movement of the elongate body towards a proximal end of the delivery system relative to the tubular member in use. This embodiment reduces the likelihood of damage occurring to the self-expanding tube due to relative movement between the tube and the elongate body during retraction.

In an embodiment, the retaining member is configured to engage detachably with the proximal region of the self-expanding tube. By applying the additional retaining force using a detachable retaining member, the self-expanding tube can be more easily released from the delivery system when deployment has been successfully completed.

In an embodiment, the proximal region of the self-expanding tube comprises a proximal engagement member, and the retaining member is configured to engage detachably with the proximal engagement member. This embodiment provides a convenient way for the retaining force to be applied to the self-expanding tube. It also provides flexibility in the mechanism by which the force is applied.

In an embodiment, the retaining member comprises a retaining tube radially positioned between the elongate body and the self-expanding tube, and at least a portion of the self-expanding tube engages inwardly with the retaining tube and outwardly with the tubular member. An engagement tube is a convenient and easily-implemented way to engage the self-expanding tube. It further reduces the likelihood of misalignment, as the retaining tube is engaged with the other cylindrical components of the delivery system.

In an embodiment, the engagement of the proximal region with the retaining member is such that the proximal region disengages from the retaining member when the proximal region is deployed beyond a distal end of the tubular member. In this embodiment, the self-expanding tube is detached automatically from the retaining member when it has been deployed sufficiently far. This further simplifies the process for releasing the stent from the remainder of the delivery system when deployment of the self-expanding tube is complete.

According to a third aspect of the invention, there is provided a delivery system for deploying a self-expanding tube into a blood vessel, configured to operate in a deployment mode and comprising a tubular member configured for insertion into the blood vessel, an elongate body extending within a lumen of the tubular member, and a self-expanding tube positioned radially between the tubular member and the elongate body, wherein the self-expanding tube comprises an elongate frame reversibly switchable from a radially expanded and longitudinally contracted state to a radially contracted and longitudinally expanded state, and a distal region of the elongate body comprises two end markers.

The two end markers on the elongate body can be used to guide the operator of the delivery system during the deployment of the self-expanding tube. The end markers are spaced apart by a predetermined distance, which can be chosen to correspond to a characteristic length that is part of the deployment process. This provides an in-situ distance measure for the operator to more clearly and precisely determine such distances, simplifying the operation of the delivery system and allowing to operator to achieve more consistent and accurate results.

In an embodiment, a distance between the end markers is equal to within 20% to the length of the self-expanding tube in the radially expanded and longitudinally contracted state. An important measure in the deployment process is the final expanded length of the self-expanding tube. This is often not straightforward to determine in-situ by the operator, due to the self-expanding tube being held in its longitudinally expanded state prior to deployment. Spacing the end markers apart by a predetermined distance which corresponds to, or is equal to within a fraction of, the final length of the self-expanding tube therefore allows the operator to determine this distance more easily during operation of the delivery system.

In an embodiment, the self-expanding tube comprises a marker located at a distal end of the self-expanding tube. Including markers on the self-expanding tube in addition to those on the guide wire improves the ability to correctly position the self-expanding tube, and to judge movement and positioning of the self-expanding tube relative to the elongate body.

In an embodiment, the tubular member comprises a marker located at a distal end of the tubular member. Including markers on the tubular member also allows the position of the elongate body and/or self-expanding tube relative to the tubular member to be ascertained more easily.

In an embodiment, the markers comprise radiopaque markers. Radiopaque markers are a particularly convenient form of marker that can easily be detected using x-ray imaging during deployment of the self-expanding tube inside the patient.

### BRIEF DESCRIPTION OF THE DRAWINGS

Embodiments of the invention will now be described, by way of example only, with reference to the accompanying drawings in which corresponding reference symbols indicate corresponding parts, and in which:

FIG. 1 is a schematic side sectional view of a distal portion of a delivery system for deploying a self-expanding tube into a blood vessel according to an embodiment of the first aspect of the invention;

FIG. 2 is a schematic end sectional view of the delivery system of FIG. 1;

FIG. 3 is a schematic side sectional view depicting a stage of deployment of a self-expanding tube in which a tubular member is longitudinally retracted relative to an elongate body;

FIG. 4 is a schematic side sectional view depicting a stage of deployment subsequent to the stage depicted in FIG. 3, in which the elongate body is retracted relative to the tubular member;

FIG. 5 depicts a stage of deployment subsequent to the stage depicted in FIG. 4, with the self-expanding tube almost fully deployed;

FIG. 6 is a schematic side sectional view of a distal portion of a delivery system for deploying a self-expanding tube into a blood vessel according to an embodiment of the second aspect of the invention; and

FIG. 7 is a schematic side sectional view of a distal portion of a delivery system for deploying a self-expanding tube into a blood vessel according to an embodiment of the third aspect of the invention.

### DETAILED DESCRIPTION

Embodiments of the present disclosure provide a delivery system 2 for deploying a self-expanding tube 6 into a blood vessel. The self-expanding tube 6 may be referred to as a stent. In a preferred embodiment the tube 6 is configured to be positioned across the opening of an aneurysmal sac to redirect blood flow away from the aneurysmal sac. The redirection of blood flow is preferably sufficient to promote thrombus formation within the sac.

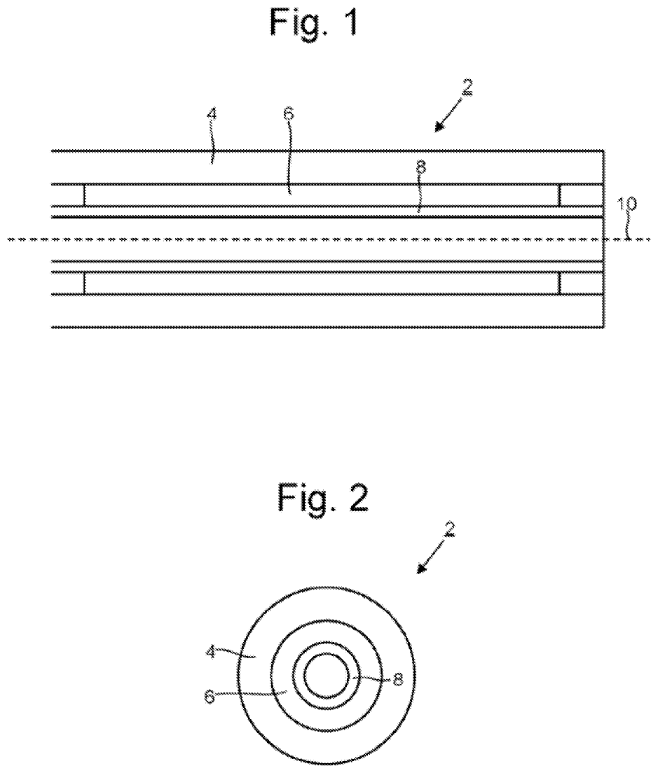

According to embodiments of the first aspect, the delivery system 2 comprises a tubular member 4 configured for insertion into the blood vessel. A distal end of the tubular member 4 is depicted in FIGS. 1 and 2. The tubular member may be referred to as a catheter. Tubular members 4 configured for such use are well known in the art of minimally invasive surgery. The tubular member 4 will typically be cylindrical and dimensioned such that its distal end can be brought to the region to be treated within the body. In the case of treating a cerebral aneurysm, the tubular member 4 will be configured so that it can be navigated to the opening of the aneurysmal sac within the vasculature of the brain. This is typically achieved by providing a flexible tubular member that can bend or flex to conform to the vasculature of a patient.

The delivery system 2 further comprises an elongate body 8 extending within a lumen of the tubular member 4. The elongate body 8 may be hollow or solid. In an embodiment, the elongate body 8 is a wire.

A self-expanding tube 6 to be deployed by the delivery system 2 is positioned radially between the tubular member 4 and the elongate body 8. The self-expanding nature of the tube 6 causes the tube 6 to engage (i.e. press) outwardly against the tubular member 4. Additionally, over at least a defined length of the tube 6, at least a portion of the tube 6 engages inwardly with the elongate body 8. Thus, over at least the defined length of the tube 6, at least a portion of the tube 6 engages (e.g. is in direct or indirect contact in the radial direction) both with the tubular member 4 and with the elongate body 8. In an embodiment the defined length is 50%, optionally 60%, optionally 70%, optionally 80%, optionally 90%, optionally 95%, optionally all or substantially all, of the length of the tube 6.

The delivery system 2 is configured to operate in a deployment mode, in which the self-expanding tube can be deployed out of the tubular member and released into a blood vessel of the patient. Deployment of the tube 6 is achieved by longitudinal retraction of the tubular member 4 relative to the elongate body 8 or longitudinally advance the elongate body 8 relative to the tubular member 4, which allows the tube 6 to self expand outwardly and leave the delivery system 2 by disengaging from the elongate body 8. The tubular member 4 and the elongate body 8 are configured such that, in the deployment mode of the delivery system 2, a first longitudinal engagement force acting between the self-expanding tube 6 and the tubular member 4 and a second longitudinal engagement force acting between the self-expanding tube 6 and the elongate body 8 are such that there is substantially no relative movement longitudinally between the elongate body 8 and any portion of the self-expanding tube 6 that remains in engagement with the elongate body 8 during deployment of the self-expanding tube 6 in use, deployment of the self-expanding tube 6 comprising longitudinal movement towards a proximal end of the delivery system 2 of the tubular member 4 relative to the elongate body 8.

To achieve the required functionality the first longitudinal engagement force between the tubular member 4 and the self-expanding tube 6, with respect to opposing deployment of the self-expanding tube 6, is weaker than the second longitudinal engagement force between the self-expanding tube 6 and the elongate body 8 at each position along the length of the self-expanding tube 6. At its simplest this may be implemented by providing a relatively low friction connection between the self-expanding tube 6 and the tubular member 4 and a relatively high friction connection between the self-expanding tube 6 and the elongate body 8. Alternatively or additionally, the outer surface of the elongate body 8 may be provided with a plurality of preformed or rigid protrusions 14. The protrusions 14 engage in use with interstices of the self-expanding tube 6, thereby increasing the longitudinal engagement force acting between the self-expanding tube 6 and the elongate body 8. The preformed protrusions may be formed by moulding or wire forming, for example. In an embodiment, the outer surface of the elongate body 8 is formed from a material which is soft above a predetermined temperature, the tube 6 is positioned against the elongate body 8 while the surface is soft, thereby forming the protrusions, and the assembly is allowed to cool until the protrusions harden and become rigid (self-supporting). In one example embodiment, the protrusions 14 are provided via a plurality of ring elements 12 that are each provided with protrusions 14 spaced regularly along the circumference of the ring element 12. It will be appreciated, however, that many other configurations could be used.

In an embodiment, the maximum obtainable first longitudinal engagement force, with respect to opposing deployment of the self-expanding tube 6 relative to the tubular member 4, is smaller than the maximum obtainable second longitudinal engagement force. This ensures that the self-expanding tube 6 will not slip relative to the tubular member 4 during deployment.

The maximum obtainable force described herein refers to the static friction between the elements when they are not moving. Once the elements of the delivery system 2 begin to move relative to one another during deployment or retraction, the frictional forces will change depending on the speed of movement and any other forces applied. Therefore, the maximum obtainable force between two elements refers to the static forces opposing a change in the state of the delivery system 2 from a state where the two elements are stationary relative to one another, to a state in which the elements move relative to one another.

The engagement between the tubular member 4 and the self-expanding tube 6 may be via direct contact between these two elements or via an intermediate element, such as a coating or other structure. The engagement between the self-expanding tube 6 and the elongate body 8 may be via direct contact between these elements or via an intermediate element such as a coating or structure.

The tubular member 4 and the elongate body 8 are further configured such that a first longitudinal engagement force acting between the self-expanding tube 6 and the tubular member 4 and a second longitudinal engagement force acting between the self-expanding tube 6 and the elongate body 8 are such that there is relative movement longitudinally between the elongate body 8 and a portion of the self-expanding tube 6 that remains in engagement with the elongate body 8 during retraction of the elongate body 8 in use after at least a portion of the self-expanding tube 6 has been deployed, retraction of the elongate body 8 comprising longitudinal movement towards a proximal end of the delivery system 2 of the elongate body 8 relative to the tubular member 4.

This can be achieved by configuring the delivery system 2 such that the maximum obtainable first longitudinal engagement force after a portion of the self-expanding tube 6 has been deployed out of the tubular member 4, with respect to opposing retraction of the self-expanding tube 6 relative to the tubular member 4, is larger than the maximum obtainable second longitudinal engagement force.

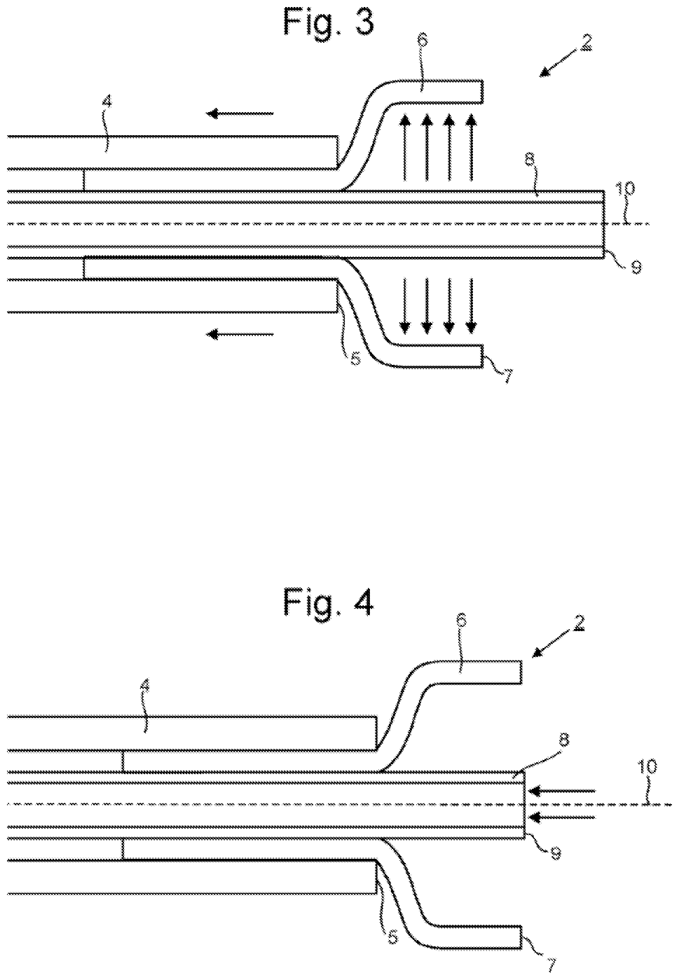

FIGS. 3 to 5 depict stages in an example deployment procedure using the delivery system 2 according to an embodiment. FIG. 3 depicts the delivery system 2 of FIGS. 1 and 2 after the tubular member 4 has been retracted longitudinally relative to the elongate body 8 (indicated by the arrows showing relative movement to the left). The relative movement can be provided by holding the elongate body 8 stationary and retracting the tubular member 4, by holding the tubular member 4 stationary and advancing the elongate body 8 or a combination of the two. As the tubular member 4 is retracted, a growing distal region of the tube 6 becomes no longer constrained radially and expands outwards. As the tube 6 expands outwards it also shortens longitudinally. This results in a distal end 9 of the elongate body 8 ending up protruding further from the tubular member 4 than a distal end 7 of the tube 6.

The protrusion of the elongate body 8 ahead of the deployed tube 6 may be undesirable. For example, the protrusion may create a risk of the elongate body 8 advancing undesirably into tissue and causing injury. This risk may be mitigated by configuring the elongate body 8 to be relatively soft and pliable. However, this may limit the range of materials that can be used for the elongate body 8, and so this solution may not be suitable in all circumstances.

FIG. 4 depicts a stage subsequent to that shown in FIG. 3 in an example deployment procedure using an embodiment of the delivery system 2. The issue of protrusion of the elongate body 8 is addressed in the embodiment by configuring the first and second longitudinal engagement forces such that, in the deployment mode of the delivery system 2, there is relative movement longitudinally between the elongate body 8 and the self-expanding tube 6 during retraction of the elongate body 8 after at least a portion of the self-expanding tube 6 has been deployed, as described above. Thereby, the longitudinal engagement forces are configured such that the possibility of relative movement between the elongate body 8 and the self-expanding tube 6 is asymmetric between deployment of the self-expanding tube 6 and retraction of the elongate body 8. In an embodiment where the elongate body 8 comprises protrusions 14, this could be achieved by providing asymmetrically-shaped protrusions. Alternative embodiments are also possible, as described further below.

The feature that the first and second longitudinal engagement forces are configured such that there is relative movement longitudinally between the elongate body 8 and the self-expanding tube 6 during retraction of the elongate body 8 after at least a portion of the self-expanding tube 6 has been deployed allows the elongate body 8, or guide wire, to move or slip relative to the self-expanding tube 6 when the elongate body 8 is retracted, such that the elongate body 8 can be retracted without also retracting the self-expanding tube 6. This in turn allows the elongate body 8 to be pulled back into the tubular member 4 and prevent the distal end 9 of the elongate body 8 from protruding more than a predetermined distance beyond the distal end 5 of the tubular member 4.

In the stage of deployment shown in FIG. 4, the elongate body 8 is retracted relative to the tubular member 4 while not substantially affecting the proportion of the self-expanding tube 6 that is deployed beyond a distal end of the tubular member 4. However, it is not essential that the self-expanding tube 6 is not retracted during retraction of the elongate body 8 in the deployment mode of the delivery system 2. It may be acceptable for the self-expanding tube 6 to be partially retracted as long as the distance by which the elongate body 8 is retracted relative to the tubular member 4 is greater than the distance by which the self-expanding tube 6 is retracted relative to the tubular member 4. This allows for movement of the elongate body 8 in a proximal direction relative to the self-expanding tube 6.

By allowing the elongate body 8 to be retracted relative to the tubular member 4 and the self-expanding tube 6, the likelihood that the elongate body 8 will cause damage to the blood vessel in which the self-expanding tube 6 is being deployed, or any other surrounding tissue, is significantly reduced.

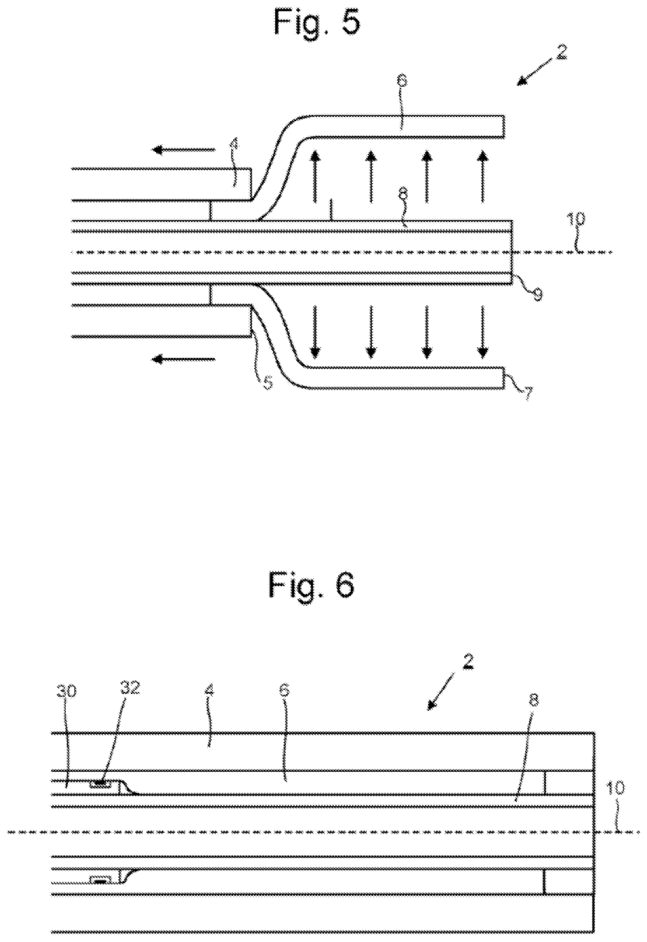

As the deployment procedure continues, as depicted in FIG. 5, more and more of the self-expanding tube 6 reaches the expanded state. However, due to the configuration of the first and second longitudinal engagement forces, as described above, the degree to which the distal end 9 of the elongate body 8 protrudes ahead of the distal end 5 of the tubular member 6 can be prevented from increasing beyond a predetermined threshold.

In an embodiment, the first longitudinal engagement force is larger, with respect to opposing retraction of the self-expanding tube 6 relative to the tubular member 4, after a portion of the self-expanding tube 6 has been deployed out of the tubular member 4 than when none of the self-expanding tube 6 has been deployed out of the tubular member 4. Having the first longitudinal engagement force change after a portion of the self-expanding tube 6 has been deployed provides greater flexibility in how the delivery system 2 can be handled. For example, it allows the self-expanding tube 6 to be freely moved back and forth with the elongate body in proximal and distal directions within the tubular member 4 prior to deployment of a portion of the self-expanding tube 6.

In an embodiment, the first longitudinal engagement force with respect to opposing retraction of the self-expanding tube 6 when none of the self-expanding tube 6 has been deployed is less than the second longitudinal engagement force. This allows the self-expanding tube 6 to be easily positioned within the tubular member 4 prior to deployment. In such an embodiment, the properties of the tubular member 4 and the elongate body 8 must be carefully designed so that the change in the first longitudinal engagement force is such that the desired asymmetry of movement of the elongate body 8 relative to the self-expanding tube 6 between deployment of the self-expanding tube 6 and retraction of the elongate body 8 is obtained after deployment of at least a portion of the self-expanding tube 6, but not before deployment of at least a portion of the self-expanding tube 6.

In an embodiment, the self-expanding tube 6 is configured to self expand from a radially contracted state to a radially expanded state in a process involving longitudinal shortening of the self-expanding tube 6 relative to a longitudinal axis of the tubular member 4, and the larger first longitudinal engagement force is achieved by engagement of a radially expanded and longitudinally contracted portion of the self-expanding tube 6 with a distal end of the tubular member 4. This mechanical engagement of the self-expanding tube 6 with the tubular member 4 is a convenient way to cause a change in the frictional forces between the self-expanding tube 6 and the tubular member 4.

In an embodiment, the maximum obtainable first longitudinal engagement force after a portion of the self-expanding tube 6 has been deployed out of the tubular member 4, with respect to opposing retraction of the self-expanding tube 6 relative to the tubular member 4, is larger than the maximum obtainable second longitudinal engagement force. This configuration of the first and second longitudinal engagement forces allows the elongate body 8 to move relative to the self-expanding tube 6.

In an embodiment, a distal end 9 of the elongate body 8 comprises a distal engagement member configured to detachably engage with the self-expanding tube 6. The distal engagement member may, for example, be used to ensure the self-expanding tube 6 remains in its radially contracted loaded position prior to deployment.

Additionally, in some situations, it may be desirable to be able to retract the self-expanding tube 6 after at least a portion of the self-expanding tube 6 has been deployed. For example, if the self-expanding tube 6 moves unexpectedly during deployment, or if the operator realises that the placement of the self-expanding tube 6 is incorrect. Therefore, in an embodiment the distal engagement member is further configured such that the maximum obtainable second longitudinal engagement force is greater than the maximum obtainable first longitudinal engagement force when the distal engagement member is engaged with the self-expanding tube 6. In such an embodiment, the distal engagement member can be used to allow the self-expanding tube 6 to be recaptured or retracted into the tubular member 4, and thereby be removed from within the blood vessel.

The delivery system 2 may be used according to preferred embodiments as part of a method of deploying a self-expanding tube into a blood vessel for the purpose of redirecting blood flow away from an aneurysmal sac. In an embodiment of such a method, deploying the self-expanding tube 6 comprises deploying a portion of the self-expanding tube 6 by longitudinally moving the tubular member 4 towards a proximal end of the delivery system 2 relative to the elongate body 8, retracting the elongate body 8 by longitudinally moving the elongate body 8 towards a proximal end of the delivery system 2 relative to the tubular member 4, and repeating the steps of deploying a portion of the self-expanding tube 6 and retracting the elongate body 8 until the self-expanding tube 6 is released from the delivery system 2 by self-expansion of the self-expanding tube 6.

Using this incremental deployment method, which is enabled by the delivery system 2 as described above, allows the elongate body 8 to be prevented from protruding more than a predetermined distance beyond the distal end of the tubular member 4 in the distal direction at any point during deployment of the self-expanding tube 6.

In an embodiment of the method, the self-expanding tube 6 is configured to self expand from a radially contracted state to a radially expanded state in a process involving longitudinal shortening of the self-expanding tube 6 relative to a longitudinal axis of the tubular member 4, and the steps of deploying a portion of the self-expanding tube 6 and retracting the elongate body 8 are performed such that at no point during the deployment of the self-expanding tube 6 does a distal end 9 of the elongate body 8 protrude beyond a distal end 5 of the self-expanding tube 6 by a distance greater than twice, optionally equal to, optionally half, the length of the self-expanding tube 6 in the radially expanded and longitudinally contracted state.

The delivery system 2 of embodiments of the present disclosure is particularly applicable to deploying self-expanding tubes 6 having low porosities, preferably porosities less than 85%, optionally less than 70%, optionally less than 60%, optionally less than 50%, when deployed in the self-expanded state. Such porosities are effective for redirecting blood flow away from an aneurysmal sac when the self-expanding tube is deployed over the opening of the aneurysmal sac. Therefore in an embodiment, the self-expanding tube 6 is configured to redirect blood flow away from an aneurysmal sac when deployed over an opening to the aneurysmal sac.

The term porosity, ρ, refers to the ratio of the surface area of open regions to the total external surface area occupied by material of the self-expanding tube 6, for example a frame of interconnecting arms. The total external surface area is the sum of the surface area of the open regions and the surface area of the regions occupied by the material of the frame. When the frame is cylindrical, the total external surface area is simply 2π·R·L, where R is the radius of the cylinder and L is the length of the cylinder.

The self-expanding tube 6 may comprise an elongate frame. The frame may comprise a shape memory alloy, for example, such as nitinol. Alternatively, the frame may comprise a stainless steel, polymer or other biocompatible material. The frame may comprise a network of interconnecting arms. The frame may be formed for example by laser cutting a hollow tube, by 3D printing, or by other techniques known in the art for manufacturing such structures. All of the interconnecting arms may be provided at the same radius and without any overlaps in the radial direction.

Consider a frame with a porosity ρ in the fully radially expanded state. If the radius and length of the frame in the fully radially expanded state are R₀ and L₀, respectively, the minimum radius Rₘᵢₙ that the frame can achieve in the radially contracted state, defined by the state in which the porosity becomes zero, is governed by

$${R}_{min} = \frac{( 1 - ρ ) {L}_{0}}{{L}_{1}} \cdot {R}_{0}$$

where L₁ is the length of the frame in the radially contracted state. This relationship assumes that elements of the frame are not allowed to overlap with each other in the radial direction.

This relationship illustrates that if the length of the frame is not allowed to change to any significant extent, the radius can only reduce by a factor of ρ. As ρ needs to be quite low (e.g. less than 80%, at least in a low porosity region, such as a region intended for positioning in use over the opening to an aneurysmal sac), this represents a significant limitation to the extent to which the tube can be narrowed for delivery to a region of interest. For example, if the porosity ρ of the frame is 20% and the length of the frame is not allowed to change during radial contraction, i.e. L₁=L₀, the frame can achieve only a maximum 20% reduction in radius. The provision of a frame that can expand longitudinally when adopting the radially contracted state is based on this understanding and allows much greater reductions in radius to be achieved. For example, if the length is allowed to double, i.e. L₁=2·L₀, the frame can achieve a 60% reduction in radius for a porosity of 20%.

In an embodiment, the longitudinal shortening of the self-expanding tube 6 comprises a shortening of at least 20%, optionally at least 30%, optionally at least 50%, optionally at least 75%, in a direction parallel to the longitudinal axis 10 of the tubular member 4, between a state in which the self-expanding tube 6 is fully within the tubular member 4 (radially) to a state in which the self-expanding tube 6 has fully left the tubular member 4 (and has expanded).

In some situations, it is necessary to recapture partially or fully the self-expanding tube 6 after at least a portion of it has been deployed into a blood vessel of the patient. This may, for example, be because the self-expanding tube 6 was initially incorrectly positioned, or if the deployed portion of the self-expanding tube 6 moves during the deployment procedure, such that it would be incorrectly positioned if deployment continued. Although these situations are uncommon if the delivery system 2 is correctly operated, allowing retraction of the self-expanding tube 6 provides a failsafe in case of difficulties, and gives peace of mind to the patient and operator of the delivery system 2 that any mistakes in deployment can be more easily rectified.

According to a second aspect, there is provided a delivery system 2 for deploying a self-expanding tube 6 into a blood vessel, comprising a tubular member 4 configured for insertion into the blood vessel, an elongate body 8 extending within a lumen of the tubular member 4, a self-expanding tube 6 positioned radially between the tubular member 4 and the elongate body 8, and a retaining member 30 configured to selectively apply a retaining force longitudinally to a proximal region of the self-expanding tube 6.

FIG. 6 is a side schematic view of an embodiment of the second aspect. The tubular member 4, self-expanding tube 6, and elongate body 8 are substantially the same as described above. The retaining member 30 allows an additional retaining force to be applied to the self-expanding tube 6. Thereby, the delivery system 2 is configured to operate in a retraction mode in which the application of the retaining force allows relative movement longitudinally between the elongate body 8 and a portion of the self-expanding tube 6 that remains in engagement with the elongate body 8 during longitudinal movement of the self-expanding tube 6 in a proximal direction relative to the elongate body 8.

This allows the self-expanding tube 6 to be retracted relative to the tubular member 4. This may be accompanied by movement of the elongate body 8 in a proximal direction relative to the tubular member 4, although this is not essential and in some embodiments the elongate body 8 and tubular member 4 are substantially stationery relative to one another during retraction of the self-expanding tube 6.

When the features of the retraction mode and retaining member 30 are provided in combination with the delivery system 2 configured to operate in a deployment mode, as described above, it is possible to reverse the asymmetry of relative movement that is possible between the self-expanding tube 6 and the elongate body 8 in the retraction mode as compared to the deployment mode. This is achieved by carefully choosing the size of the retaining force applied using the retaining member 30 relative to the first and second longitudinal engagement forces. The retaining force may take the form of tension applied to the proximal region of the self-expanding tube 6 by the retaining member 30 in a proximal direction.

In an embodiment, in the retraction mode, the application of the retaining force is such that there is substantially no relative movement longitudinally between the elongate body 8 and any portion of the self-expanding tube 6 that remains in engagement with the elongate body 8 during longitudinal movement of the elongate body 8 towards a proximal end of the delivery system 2 relative to the tubular member 4 in use.

In such an embodiment, the lack of movement of the elongate body 8 relative to the self-expanding tube 6 reduces the chance of damage occurring to the self-expanding tube 6 as a result of abrasion, or any part of the self-expanding tube 6 being deformed in an unintended manner. It also reduces the size of the retaining force that must be applied to the self-expanding tube 6 by the retaining member 30, as it will not be necessary to overcome the second longitudinal engagement force in addition to the first longitudinal engagement force in order to cause the self-expanding tube 6 to move in a proximal direction. This further reduces the risk of damage to the self-expanding tube 6.

In an embodiment, in the retraction mode, the sum of the retaining force and the first longitudinal engagement force is larger, with respect to opposing deployment of the self-expanding tube 6 relative to the tubular member 4, than the maximum obtainable second longitudinal engagement force.

This embodiment allows the elongate body 8 to move in a distal direction relative to the self-expanding tube 6 in the retraction mode. In the case where the delivery system 2 is such that the elongate body 8 is kept from protruding more than a predetermined distance beyond the end of the tubular member 4, retracting the self-expanding tube 6 and elongate body 8 together at the same rate could result in a portion of the self-expanding tube 6 not being in engagement with the elongate body 8 inside the tubular member 4. This is because the self-expanding tube 6 will expand longitudinally as it is retracted into the tubular member 4 and contracted radially. The lack of support of the self-expanding tube 6 by the elongate body 8 inside the tubular member 4 may result in damage to the self-expanding tube 6, which would make redeployment of the tube difficult or dangerous to the patient. Allowing the elongate body 8 to move distally relative to the tube 6 can be used to ensure that the tube 6 is always properly engaged with the elongate body 8 inside the tubular member 4, while maintaining the advantage of preventing the elongate body 8 protruding too far beyond the distal end of the tubular member 4.

In an embodiment, in the retraction mode, the maximum obtainable first longitudinal engagement force is smaller, with respect to opposing retraction of the self-expanding tube 6 relative to the tubular member 4, than the sum of the retaining force and the second longitudinal engagement force. This embodiment represents a choice of the relative size of the forces that allows the self-expanding tube 6 to be retracted relative to the tubular member 4, as described above.

In an embodiment, the retaining member 30 is configured to engage detachably with the proximal region of the self-expanding tube 6. This embodiment provides the advantage that the deployment of the self-expanding tube 6 is more easily completed. The detachable engagement may be provided through any suitable means, for example, hooks on the retaining member 30 configured to engage with the structure of the self-expanding tube 6. Other alternatives include electrolytic attachment, where the retaining member 30 and self-expanding tube 6 are engaged via a dissolvable metallic element that can be dissolved once the self-expanding tube 6 has been sufficiently deployed.

In an embodiment, the proximal region of the self-expanding tube 6 comprises a proximal engagement member 32, and the retaining member 30 is configured to engage detachably with the proximal engagement member 32. In the specific example shown in FIG. 6, two proximal engagement members 32 are provided, however in general any number of proximal engagement members 32 may be provided.

The proximal engagement members 32 may engage with the retaining member 30 via any suitable mechanism. For example, in the embodiment of FIG. 6, the proximal engagement members 32 comprise solid blocks which engage with recesses in the retaining member 30. However, other mechanisms are possible, for example a hook-shaped proximal engagement member that engages with a loop on the retaining member 30, or vice-versa. Detachment of the proximal engagement member 32 from the retaining member 30 may be controlled directly by the operator through the provision of an actuation mechanism controlled at a proximal end of the delivery system 2. Alternatively, as described further below, the detachment may be substantially automatic.

In an embodiment, the retaining member 30 comprises a retaining tube radially positioned between the elongate body 8 and the self-expanding tube 6, and at least a portion of the self-expanding tube 6 engages inwardly with the retaining tube and outwardly with the tubular member 4. Providing the retaining member 30 in the form of a retaining tube, as shown in FIG. 6, provides the advantage that the retaining member 30 is securely and consistently positioned relative to the other components of the delivery system 2. Ensuring that the self-expanding tube 6 engages inwardly with the retaining tube provides a convenient way to allow the retaining member 30 to engage with the self-expanding tube 6 and apply the retaining force.

In an embodiment, the engagement of the proximal region of the self-expanding tube 6 with the retaining member 30 is such that the proximal region disengages from the retaining member 30 when the proximal region is deployed beyond a distal end of the tubular member 4. This embodiment is advantageous because it means that no additional action is required by the operator to complete deployment of the self-expanding tube 6, which simplifies the deployment process and reduces the chance of error. A variety of different mechanisms may be used to provide this feature. In the embodiment shown in FIG. 6, the retaining tube as described above, in combination with the proximal engagement members 32 means that self-expansion of the proximal region of the self-expanding tube 6 will cause the proximal engagement members 32 to disengage from the retaining member 30 once the proximal region of the self-expanding tube is no longer outwardly constrained by the tubular member 4. In an alternative embodiment, a dissolvable element connects between the self-expanding tube 6 and the retaining member 30 is used, where the dissolvable element dissolves on exposure to the environment of the blood vessel, and releases the self-expanding tube 6 from the delivery system 2.

When using a self-expanding tube 6 that is reversibly switchable from a radially expanded and longitudinally contracted state to a radially contracted and longitudinally expanded state, it is helpful during deployment to be able to determine how far to deploy the self-expanding tube 6 in a single action or movement of the elongate body 8. This is because the length of a portion of compressed self-expanding tube 6 before deployment does not correspond to the length of the same portion once deployed. This makes it difficult to keep track of how much of the tube 6 has been deployed in a single action.

In particular, when using an incremental deployment mechanism such as that described above, where the elongate body 8 is alternatively deployed and then retracted relative to the self-expanding tube 6, a balance needs to be made between two factors. The first is not extending the elongate body 8 too far beyond the end of the tubular member 4 before retracting the elongate body 8 again relative to the self-expanding tube 6. Extending the elongate body 8 too far would risk damaging the blood vessel, as described earlier. The second is not extending the elongate body 8 by too short a distance beyond the end of the tubular member 4 in each step, which would result in an excessively large number of deployment/retraction cycles to deploy the self-expanding tube 6. A large number of deployment/retraction cycles increases the complexity and difficulty of the deployment procedure, thereby increasing the chance of user error. An optimal deployment distance for each iteration of the cycle will find a balance between these two factors. Placing markers on the elongate body 8 can provide a guide to the operator of an optimal distance by which to deploy the elongate body 8 in each deployment/retraction cycle.

Markers on the elongate body 8 can also be used for other purposes, such as proper positioning of the self-expanding tube 6 during deployment. It is beneficial for the operator to be able to determine during deployment where the self-expanding tube 6 should be deployed to properly cover the neck of an aneurysm. In many prior art devices, it is not possible to accurately predict the final length of the self-expanding tube 6 because the degree of longitudinal contraction is dependent on the degree of radial expansion, which itself is dependent on the exact size and shape of the blood vessel into which the self-expanding tube 6 is deployed. This is particularly true when using self-expanding tubes 6 comprised largely of a wire mesh. Therefore, if markers are included on the elongate body 8, they are usually only included on the elongate body 8 at a distal end, corresponding to the position of the distal end of the self-expanding tube 6 at the start of the deployment process. This often provides insufficient guidance to the user for placing the self-expanding tube 6, as the position of the distal end is not a good indicator of the final position of the proximal end, so the self-expanding tube 6 may easily be placed incorrectly, necessitating time-consuming and potentially difficult retraction and redeployment of the stent.

However, when using a design of self-expanding tube 6 such as described herein, the longitudinal contraction and radial expansion that occur on deployment are substantially independent, and the final length of the tube 6 is more consistent and predictable. This makes it possible to include markers spaced apart by a distance on the elongate body 8 representative of the final, deployed length of the self-expanding tube 6. These markers aid in positioning the self-expanding tube 6 during deployment, so that it can be ensured that the self-expanding tube 6 will cover the aneurysm neck properly, and without risk of later movement of the self-expanding tube 6.

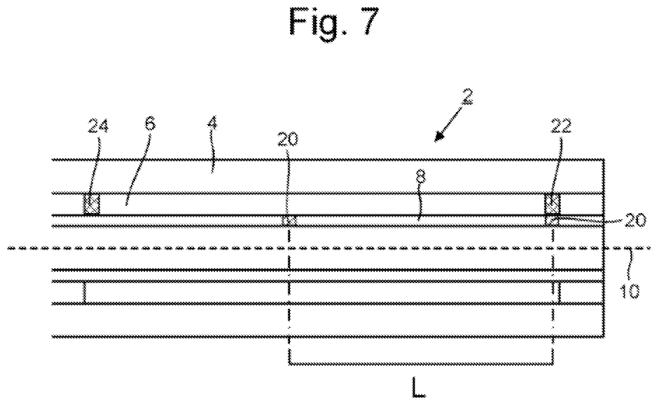

FIG. 7 depicts a delivery system 2 for deploying a self-expanding tube 6 into a blood vessel, comprising a tubular member 4 configured for insertion into the blood vessel, an elongate body 8 extending within a lumen of the tubular member 4, and a self-expanding tube 6 positioned radially between the tubular member 4 and the elongate body 8, wherein the self-expanding tube 6 comprises an elongate frame reversibly switchable from a radially expanded and longitudinally contracted state to a radially contracted and longitudinally expanded state, and a distal region of the elongate body 8 comprises two end markers 20.

In an embodiment, a distance L between the end markers 20 is equal to within 20%, optionally to within 10%, optionally to within 5%, to the length of the self-expanding tube 6 in the radially expanded and longitudinally contracted state. Due to the fact that the self-expanding tube 6 contracts longitudinally on deployment, the distance L between the end markers 20 prior to deployment of the self-expanding tube is substantially less than the length of the self-expanding tube 6 in the radially contracted and longitudinally expanded state. This feature may help with positioning of the self-expanding tube 6 during deployment. In an embodiment, the optimal deployment distance for a single deployment/retraction cycle is between 10% and 90%, optionally between 25% and 75%, of the final length of the self-expanding tube 6 in its longitudinally contracted and radially expanded state.

In an embodiment, a distance L between the end markers 20 is equal to within 2 mm, optionally to within 1 mm, optionally to within 0.5 mm, to the length of the self-expanding tube 6 in the radially expanded and longitudinally contracted state. In the case where the self-expanding tube 6 is used to treat brain aneurysms, this represents a suitable spacing for the size of self-expanding tube 6 suitable for treating brain aneurysms.

In an embodiment such as that shown in FIG. 7, the self-expanding tube 6 comprises a marker 22 located at a distal end 7 of the self-expanding tube 6. In an embodiment, the self-expanding tube 6 further comprises a marker 24 located at a proximal end of the self-expanding tube 6. These markers 22, 24 make it easier for the operator to determine where the ends of the self-expanding tube 6 are during the deployment process, and can be aligned with the end markers 20 on the elongate body 8.

The use of end markers 20 is particularly advantageous in embodiments of the delivery system 2 described earlier, where the first and second longitudinal engagement forces are configured such that the elongate body 8 can be retracted relative to the self-expanding tube 6. This is because they provide a reference to allow the operator to extend a consistent and/or optimal length of the self-expanding tube 6 in each deployment/retraction cycle. In an embodiment, the tubular member 4 comprises a marker located at a distal end of the tubular member 4. The marker on the tubular member 4 can be used as a reference relative to the markers on the elongate body 8, so that the elongate body 8 is retracted to the same position relative to the tubular member 4, and deployed by the same distance relative to the tubular member 4 in each deployment/retraction cycle.

The elongate body 8 can also be repositioned throughout the deployment process, so that the end markers 20 can be used to monitor whether movement of the delivery system 2 during deployment has affected the final position of the self-expanding tube 6. In an embodiment where the self-expanding tube 6 comprises one or more markers 22, 24, the end markers 20 on the elongate body 8 can be aligned with the markers on the self-expanding tube 6 and used as a ruler to check that the deployment of the self-expanding tube 6 will place it correctly relative to the aneurysm.

In an embodiment, the markers 20, 22, 24 comprise radiopaque markers. X-ray imaging is commonly used to monitor the deployment of a stent into a blood vessel, and so radiopaque markers are particularly suitable for devices designed for use in such procedures. Further, to improve visibility of the delivery system 2 for the operator during deployment, the elongate body 8 may comprise a radiopaque wire, and/or at least part of the self-expanding tube may be composed of radiopaque wire. Radiopaque wire can comprise wire made, wholly or in part, from a material chosen for its opacity to the type of radiation used for imaging.

The markers 20, 22, 24 may be in the form of spots or bands placed on or inside the elongate body 8 and/or self-expanding tube 6. Alternatively, the markers may comprise rings around a circumference of the elongate body 8 or self-expanding tube 6. The use of ring-shaped markers may be advantageous in allowing the markers to be more clearly seen regardless of the relative orientation of the delivery system 2 and the imaging system used to monitor the procedure.

In an embodiment where the self-expanding tube 6 comprises a marker 24 placed at a proximal end of the self-expanding tube 6, the marker 24 may also function as a proximal engagement member 32.

The delivery system comprising end markers 20 is suitable for use in a method of deploying a self-expanding tube 6 into a blood vessel, wherein deploying the self-expanding tube 6 comprises deploying a portion of the self-expanding tube 6 by longitudinally moving the tubular member 4 towards a proximal end of the delivery system 2 relative to the elongate body 8 (or equivalently longitudinally moving the elongate body 8 in a distal direction relative to the tubular member 4), retracting the elongate body 8 by longitudinally moving the elongate body 8 towards a proximal end of the delivery system 2 relative to the tubular member 4, and repeating the steps of deploying a portion of the self-expanding tube 6 and retracting the elongate body 8 until the self-expanding tube 6 is released from the delivery system 2 by self-expansion of the self-expanding tube 6, wherein during at least one repetition of the step of deploying a portion of the self-expanding tube 6, the self-expanding tube 6 is deployed by a distance equal to within 25%, optionally within 15%, optionally within 10%, optionally within 5%, to the distance between the end markers 20. In this method, the end markers 20 provide the function of acting as a reference for the operator, such that the self-expanding tube 6 is deployed by a consistent distance. In an embodiment, the distance L between the markers 20 is chosen to correspond to an optimal distance. Corresponding to the optimal distance may entail being equal to, or equal to within, for example 10% of, a distance which balances the two factors described above, i.e. avoiding extending the elongate body 8 too far beyond the distal end of the tubular member 4 with each repetition of the deployment step, and avoiding too large a number of deployment steps being necessary to deploy the self-expanding tube 6.

In an embodiment, the optimal distance is related to a length of the self-expanding tube in the radially expanded and longitudinally contracted state. For example, as suggested above for the distance between the end markers, the optimal distance may be equal to within a predetermined percentage, for example between 25%-75%, of the length of the self-expanding tube in the radially expanded and longitudinally contracted state.

In one embodiment, either or both of a composition and surface texture of the inner surface of the tubular member 4 is or are arranged to be uniform over a length in which the tubular member 4 is in contact with the self-expanding tube 6. Optionally, a low friction coating may be provided on the inner surface of the tubular member 4.

The elongate body 8 may also be configured such that either or both of a composition and surface texture of the outer surface of the elongate body 8 is uniform over a length in which the elongate body 8 is in contact with the self-expanding tube 6. Even where the surface is uniform, it would be straightforward for the skilled person to arrange for the frictional engagement force between the self-expanding tube 6 and the elongate body 8 to be higher than the frictional engagement force provided by the tubular member 4 with respect to opposing deployment of the self-expanding tube 6, for example by providing a suitable high friction coating or surface roughening.

We claim:

1. A delivery system for deploying a self-expanding tube into a blood vessel, comprising:
a tubular member configured for insertion into the blood vessel;
an elongate body extending within a lumen of the tubular member; and
a self-expanding tube positioned radially between the tubular member and the elongate body,
wherein the delivery system is configured to operate in a deployment mode in which a first longitudinal engagement force acting between the self-expanding tube and the tubular member and a second longitudinal engagement force acting between the self-expanding tube and the elongate body are such that:
there is substantially no relative movement longitudinally between the elongate body and any portion of the self-expanding tube that remains in engagement with the elongate body during deployment of the self-expanding tube in use, deployment of the self-expanding tube comprising longitudinal movement towards a proximal end of the delivery system of the tubular member relative to the elongate body; and
there is relative movement longitudinally between the elongate body and a portion of the self-expanding tube that remains in engagement with the elongate body during retraction of the elongate body in use after at least a portion of the self-expanding tube has been deployed, retraction of the elongate body comprising longitudinal movement towards a proximal end of the delivery system of the elongate body relative to the tubular member.

2. The delivery system of claim 1, configured such that:
the first longitudinal engagement force is larger, with respect to opposing retraction of the self-expanding tube relative to the tubular member, after a portion of the self-expanding tube has been deployed out of the tubular member than when none of the self-expanding tube has been deployed out of the tubular member.

3. The delivery system of claim 2, wherein:
the self-expanding tube is configured to self expand from a radially contracted state to a radially expanded state in a process involving longitudinal shortening of the self-expanding tube relative to a longitudinal axis of the tubular member; and
the larger first longitudinal engagement force is achieved by engagement of a radially expanded and longitudinally contracted portion of the self-expanding tube with a distal end of the tubular member.

4. The delivery system of claim 2, configured such that the maximum obtainable first longitudinal engagement force after a portion of the self-expanding tube has been deployed out of the tubular member, with respect to opposing retraction of the self-expanding tube relative to the tubular member, is larger than the maximum obtainable second longitudinal engagement force.

5. The delivery system of claim 1, configured such that the maximum obtainable first longitudinal engagement force, with respect to opposing deployment of the self-expanding tube relative to the tubular member, is smaller than the maximum obtainable second longitudinal engagement force.

6. The delivery system of claim 1, wherein a distal end of the elongate body comprises a distal engagement member configured to detachably engage with the self-expanding tube.

7. The delivery system of claim 6, wherein the distal engagement member is further configured such that the maximum obtainable second longitudinal engagement force is greater than the maximum obtainable first longitudinal engagement force when the distal engagement member is engaged with the self-expanding tube.

8. The delivery system of claim 1, wherein over at least 50% of the length of the self-expanding tube, at least a portion of the self-expanding tube engages outwardly with the tubular member and inwardly with the elongate body.

9. The delivery system of claim 1, configured such that either or both of a composition and surface texture of the inner surface of the tubular member is uniform over a length in which the tubular member is in contact with the self-expanding tube.

10. The delivery system of claim 1, configured such that either or both of a composition and surface texture of the outer surface of the elongate body is uniform over a length in which the elongate body is in contact with the self-expanding tube.

11. The delivery system of claim 1, further comprising a retaining member configured to selectively apply a retaining force longitudinally to a proximal region of the self-expanding tube, the delivery system further configured to operate in a retraction mode in which the application of the retaining force allows relative movement longitudinally between the elongate body and a portion of the self-expanding tube that remains in engagement with the elongate body during longitudinal movement of the self-expanding tube in a proximal direction relative to the elongate body.

12. A delivery system for deploying a self-expanding tube into a blood vessel, comprising:
a tubular member configured for insertion into the blood vessel;
an elongate body extending within a lumen of the tubular member;
a self-expanding tube positioned radially between the tubular member and the elongate body; and
a retaining member configured to selectively apply a retaining force longitudinally to a proximal region of the self-expanding tube,
wherein the delivery system is configured to operate in a retraction mode in which the application of the retaining force allows relative movement longitudinally between the elongate body and a portion of the self-expanding tube that remains in engagement with the elongate body during longitudinal movement of the self-expanding tube in a proximal direction relative to the elongate body.

13. The delivery system of claim 12, wherein, in the retraction mode, the application of the retaining force is such that there is substantially no relative movement longitudinally between the elongate body and any portion of the self-expanding tube that remains in engagement with the elongate body during longitudinal movement of the elongate body towards a proximal end of the delivery system relative to the tubular member in use.

14. The delivery system of claim 12, wherein, in the retraction mode, the sum of the retaining force and a first longitudinal engagement force acting between the self-expanding tube and the tubular member is larger, with respect to opposing deployment of the self-expanding tube relative to the tubular member, than a maximum obtainable second longitudinal engagement force acting between the self-expanding tube and the elongate body.

15. The delivery system of claim 12, wherein, in the retraction mode, a maximum obtainable first longitudinal engagement force acting between the self-expanding tube and the tubular member is smaller, with respect to opposing retraction of the self-expanding tube relative to the tubular member, than the sum of the retaining force and a second longitudinal engagement force acting between the self-expanding tube and the elongate body.

16. The delivery system of claim 12, wherein the retaining member is configured to engage detachably with the proximal region of the self-expanding tube.

17. The delivery system of claim 16, wherein the proximal region of the self-expanding tube comprises a proximal engagement member, and the retaining member is configured to engage detachably with the proximal engagement member.

18. The delivery system of claim 12, wherein the retaining member comprises a retaining tube radially positioned between the elongate body and the self-expanding tube, and at least a portion of the self-expanding tube engages inwardly with the retaining tube and outwardly with the tubular member.

19. The delivery system of claim 16, wherein the engagement of the proximal region with the retaining member is such that the proximal region disengages from the retaining member when the proximal region is deployed beyond a distal end of the tubular member.

20. A delivery system for deploying a self-expanding tube into a blood vessel, configured to operate in a deployment mode and comprising:
a tubular member configured for insertion into the blood vessel;
an elongate body extending within a lumen of the tubular member; and
a self-expanding tube positioned radially between the tubular member and the elongate body, wherein:
the self-expanding tube comprises an elongate frame reversibly switchable from a radially expanded and longitudinally contracted state to a radially contracted and longitudinally expanded state; and
a distal region of the elongate body comprises two end markers.

21. The delivery system of claim 20, wherein a distance between the end markers is either or both of (i) equal to within 20% to the length of the self-expanding tube in the radially expanded and longitudinally contracted state; and (ii) equal to within 2 mm to the length of the self-expanding tube in the radially expanded and longitudinally contracted state.

22. The delivery system of claim 20, wherein the self-expanding tube comprises a marker located at a distal end of the self-expanding tube.

23. The delivery system of claim 22, wherein the self-expanding tube further comprises a marker located at a proximal end of the self-expanding tube.

24. The delivery system of claim 20, wherein the tubular member comprises a marker located at a distal end of the tubular member.

25. A method of deploying a self-expanding tube into a blood vessel comprising operating the delivery system of claim 1 in the deployment mode to deploy the self-expanding tube, wherein deploying the self-expanding tube comprises:
deploying a portion of the self-expanding tube by longitudinally moving the tubular member towards a proximal end of the delivery system relative to the elongate body;
retracting the elongate body by longitudinally moving the elongate body towards a proximal end of the delivery system relative to the tubular member; and
repeating the steps of deploying a portion of the self-expanding tube and retracting the elongate body until the self-expanding tube is released from the delivery system by self-expansion of the self-expanding tube.

26. The method of claim 25, wherein:
the self-expanding tube is configured to self expand from a radially contracted state to a radially expanded state in a process involving longitudinal shortening of the self-expanding tube relative to a longitudinal axis of the tubular member; and
the steps of deploying a portion of the self-expanding tube and retracting the elongate body are performed such that at no point during the deployment of the self-expanding tube does a distal end of the elongate body protrude beyond a distal end of the self-expanding tube by a distance greater than 2 times the length of the self-expanding tube in the radially expanded and longitudinally contracted state.

27. A method of deploying a self-expanding tube into a blood vessel comprising operating the delivery system of claim 20 in the deployment mode to deploy the self-expanding tube, wherein deploying the self-expanding tube comprises:
deploying a portion of the self-expanding tube by longitudinally moving the elongate body towards a distal end of the delivery system relative to the tubular member;
retracting the elongate body by longitudinally moving the elongate body towards a proximal end of the delivery system relative to the tubular member; and
repeating the steps of deploying a portion of the self-expanding tube and retracting the elongate body until the self-expanding tube is released from the delivery system by self-expansion of the self-expanding tube, wherein:
during at least one repetition of the step of deploying a portion of the self-expanding tube, the self-expanding tube is deployed by a distance equal to within 50% to the distance between the end markers.
